(19) United States
(12) Patent Application Publication
(10) Pub. No.: US 2025/0177465 A1
(43) Pub. Date: Jun. 5, 2025

(54) TRADITIONAL CHINESE MEDICINE COMPOSITION AND PREPARATION METHOD AND APPLICATION THEREOF

(71) Applicant: MEDONCARE PHARMACEUTICAL CO., LTD, Changsha, Hunan (CN)

(72) Inventors: Xiaozhong LIU, Changsha, Hunan (CN); Wenjuan XIA, Changsha, Hunan (CN); Hujuan ZHANG, Changsha, Hunan (CN); Hainan XIU, Changsha, Hunan (CN); Qifeng JIN, Changsha, Hunan (CN)

(21) Appl. No.: 18/724,860

(22) PCT Filed: Dec. 29, 2022
(86) PCT No.: PCT/CN2022/143364

§ 371 Date: Jan. 17, 2025

(30) Foreign Application Priority Data
Dec. 29, 2021 (CN) 202111645670.5

(51) Int. Cl. A61K 36/42; A61K 31/05; A61K 31/12; A61K 31/198; A61K 31/205; A61K 31/216; A61K 31/4172; A61K 31/702; A61K 36/232; A61K 36/28; A61K 36/346; A61K 36/484; A61K 36/536; A61K 36/54; A61K 36/61; A61K 36/67; A61K 36/73; A61K 36/744; A61K 36/752; A61K 36/78; A61K 36/899; A61K 47/02; A61K 47/10; A61K 47/32; A61K 47/36; A61K 47/38; A61P 11/00; A61P 11/04; A61P 31/14

(52) U.S. Cl. CPC A61K 36/42; A61K 31/05; A61K 31/12; A61K 31/198; A61K 31/205; A61K 31/216; A61K 31/4172; A61K 31/702; A61K 36/232; A61K 36/28; A61K 36/346; A61K 36/484; A61K 36/536; A61K 36/54; A61K 36/61; A61K 36/67; A61K 36/73; A61K 36/744; A61K 36/752; A61K 36/78; A61K 36/899; A61K 47/02; A61K 47/10; A61K 47/32; A61K 47/36; A61K 47/38; A61P 11/00; A61P 11/04; A61P 31/14; A61K 2236/15; A61K 2236/19; A61K 2236/331; A61K 2236/333; A61K 2236/37; A61K 2236/51

(57) ABSTRACT

A traditional Chinese medicine composition for treating abnormal lung function and laryngitis caused by the influenza virus; the traditional Chinese medicine composition comprising as a primary formula of active ingredients seven medicinal/edible source materials composed of: Siraitia grosvenorii, Cinnamomum cassia, Prunella vulgaris, Houttuynia cordata, Platycodon grandiflorus, Gardenia jasminoides and Glycyrrhiza uralensis. The foundation of the primary formula is supplemented by a secondary formula of seven medicinal/edible source materials consisting of: Citrus reticulata (mandarin orange), Eriobotrya japonica leaves, Angelica dahurica, Arctium lappa root, Piper nigrum, Phragmites australis root and Syzygium aromaticum, and different preparations are prepared. The traditional Chinese medicine combined formula can be a combined formula of source herbs, and can likewise be a combined formula of extracts thereof, and can likewise be a combined formula of source herbs and extracts. The traditional Chinese medicine composition has a significant therapeutic effect on symptoms such as sore throat, itchy throat cough, swelling and pain in the throat, pharyngeal discomfort, etc. resulting from influenza-caused pneumonia such as abnormal lung function and difficulty breathing, cough, lung nodules, ground glass opacity in the lungs, pulmonary fibrosis, interstitial lung disease and laryngitis, and has a certain relieving, progression-stopping and disease condition control and treatment effects on chronic obstructive emphysema.

### TECHNICAL FIELD

The present application relates to a traditional Chinese medicine composition for lung function abnormality and pharyngitis caused by influenza virus and a preparation method and application/use thereof, belonging to the technical field of medicine.

### BACKGROUND

Influenza virus pneumonia is an infectious disease of the lungs caused by the invasion of the lower respiratory tract by the influenza virus, which can be caused by direct invasion of the virus itself, or by secondary bacterial infection. Influenza virus pneumonia is easy to occur in patients with underlying lung and heart diseases, pregnant women, infants, the elderly or immunodeficient people; it is manifested as with influenza symptoms at early stage, and the fatality rate is higher in patients with rapid progression.

After the virus infects lungs, it directly causes an inflammatory reaction in the lungs, resulting in a large amount of tissue fluid exudation, leading to pulmonary edema. Under CT examination of lung lesions, there exist glass shadows or veins, pulmonary fibrosis, alveoli filled with fluid, and emphysema. The specific symptoms are dyspnea and persistent cough.

The replication and spread of the virus in the human body causes a series of overreactions of the human immune system, causing an inflammatory storm. In addition to causing severe damage to the lungs, the virus can also affect multiple major organs of the body, causing rapid deterioration in some patients. It can then lead to multiple organ failure, a decline in liver and kidney function, and gastrointestinal tract dysfunction.

The novel coronavirus is commonly referred to as the 2019 novel coronavirus (2019-NCoV, named by the WHO in January 2020; SARS-CoV-2, named by the International Committee on Taxonomy of Viruses, 11 Feb. 2020). 2019-NCOV is a newly emerged variant of coronavirus, so current clinical studies are preliminary and phased. The incubation period of the virus is about 3-7 days on average, and the longest is not more than 14 days. Most patients present with the following respiratory symptoms, common clinical manifestations include fever, limb weakness, dry cough, etc. Other manifestations include nasal congestion, runny nose, headache, sore throat, coughing up blood, coughing up phlegm, or diarrhea. Some patients only show low fever, mild fatigue, etc., without pneumonia. There are also some patients without any clinical manifestations. Severe viral infections can lead to a variety of complications including acute respiratory distress syndrome (ARDS), septic shock, intractable metabolic acidosis, acute myocardial injury, and coagulation dysfunction.

SARS-CoV-2 attacks the human body, simultaneously attacking the lungs, heart, liver, kidney, stomach and nervous system, causing multiple organ failure and damage. Once human organs are damaged, irreversible damage occurs. After the epidemic, it will become the most severe reality for thousands of infected people how to recover as soon as possible and how to reduce and eliminate the damage of inflammatory factors on human tissues and organs, in the process of treatment and rehabilitation, especially in the late recovery of severe and critically ill patients. After the virus infects the lungs, it directly causes the inflammatory reaction of the lungs, resulting in a large amount of tissue fluid exudation, causing pulmonary edema. Under CT examination of lung lesions, there exist glass shadows or veins, pulmonary fibrosis, alveoli filled with fluid, and emphysema. The specific symptoms are dyspnea and persistent cough. Therefore, rehabilitation treatment for the prognosis of those infected with the novel coronavirus is crucial and should not be ignored. Common sequelae include pulmonary fibrosis, ground-glass opacity, interstitial lung lesions, low oxygenation, and dyspnea.

Pulmonary fibrosis is a major category of end-stage changes of lung diseases characterized by fibroblast proliferation and accumulation of a large number of extracellular matrixes, accompanied by inflammatory damage and tissue structure destruction. That is, normal alveolar tissue is damaged and abnormally repaired, resulting in structural abnormalities (scar formation). Pulmonary fibrosis seriously affects the human respiratory function, and is manifested as dry cough, progressive dyspnea (consciousness of insufficient gas), and with the aggravation of the disease and lung damage, the patient's respiratory function continues to deteriorate.

Generally speaking, pulmonary ground-glass opacity is that under the chest CT examination lung parenchyma shows the cloud dense shadow, single, or multiple, small as 2-3 mm, large even more than 2 cm, wherein sometimes trachea or blood vessels can be seen. The pathological causes may be the thickening of pulmonary interstitial due to inflammation, edema, fibrous tissue hyperplasia and other reasons after virus infection, and incomplete packing of cells and fluid in the alveolar cavity.

Interstitial lung diseases are a group of diffuse lung diseases that mainly involve the interstitial lung and alveolar cavity, resulting in the loss of alveolar-capillary functional units. The clinical manifestations are progressive increased dyspnea, restricted ventilation dysfunction with decreased diffusion function, hypoxemia, and radiographic diffuse lesions of both lungs, which may progress to diffuse pulmonary fibrosis, honeycomb lung, and eventual conduction. It leads to respiratory failure and death, which is mainly treated with drugs at present.

When the virus invades the lungs, it causes damage to the lung tissue and affects gas exchange, resulting in decreased oxygen saturation, which can lead to fluid filling or stiffness in the lungs, increased carbon dioxide levels, shortness of breath, and even difficulty breathing.

Chronic obstructive pulmonary disease (COPD) is a group of lung diseases characterized by airflow restriction, which is not completely reversible but develops progressively and mainly affects the lungs. The exact cause of COPD is unknown, but it is thought to be associated with abnormal inflammatory responses to harmful gases and particles, with a high rate of disability and fatality. The main pathological changes are chronic bronchitis and emphysema. The epithelial cells of the bronchial mucosa are denaturated, necrotic and ulcerated. The cilia are lodging, shortened, uneven, adherent, and partially detached. In remission, mucosal epithelium repair, hyperplasia, squamous metaplasia, and granuloma formation occur. Goblet cells increase in number and hypertrophy, secretion is hyperactive, and intracavitary secretions remain. The basement membrane becomes thick and necrotic. Hyperplasia and hypertrophy occur in bronchial glandular, and the ratio of glandular hypertrophy to bronchial wall thickness is often greater than 0.55-0.79 (normal less than 0.4). There are various inflammatory cells infiltrating the bronchial wall at all levels, mainly neutrophils and lymphocytes. A large number of neutrophils can be seen in acute attacks, and in the severe cases there are suppurative inflammation, mucosal congestion, edema, degeneration, necrosis and ulcer formation, and the hyperplasia of the granulation tissue of the base and the structured fibrous tissue leading to lumen stenosis. Inflammation leads to repeated injury-repair of the airway wall, which leads to remodeling of trachea structure, increased collagen content and scar formation. These pathological changes are one of the main pathological bases of airflow restriction in COPD. The pathological changes of emphysema can be manifested as the lung hyperexpansion and reduced elasticity. The appearance is gray or pale, and multiple bullae of different sizes can be seen on the surface. Microscopic examination shows thinning of the alveolar walls, enlargement, rupture, or bullosa formation of the alveolar cavity, reduced blood supply, and destruction of the elastic fiber network. There is inflammatory cell infiltration in the bronchiolar wall, hyperplasia and hypertrophy of mucous glands and goblet cells in the tube wall, damage of ciliary epithelium and reduction of cilia. In some cases, the lumen is slender and narrow or twisted and dilated, and the sputum remains in the lumen. The clinical manifestations are chronic cough, shortness of breath or dyspnea, wheezing and chest tightness.

Pharyngitis is divided into acute and chronic pharyngitis. Acute pharyngitis is often caused by viruses, followed by bacteria. It is most common in winter and spring. It is more secondary to acute rhinitis, acute sinusitis, acute tonsillitis, and often measles, influenza, scarlet fever and other infectious diseases complications. Chronic pharyngitis, mainly because incomplete treatment to the acute pharyngitis and repeated episodes acute pharyngitis turn to be chronic, or because of stimulation to pharyngitis by various nasal diseases, nasal obstruction, long-term open mouth breathing, as well as physical and chemical factors, neck radiation treatment, is manifested as pharyngitis symptoms and the significant systemic symptoms which are, fever and fear of cold, headache, loss of appetite, aching limbs and so on. Chronic simple pharyngitis is a chronic inflammation of the pharynx mucosa, submucosa and its lymphatic tissue. The lesion is mainly in the mucosal layer, manifested as diffuse congestion of the pharyngeal mucosa, and there is more lymphocyte infiltration around the blood vessels, and leukocyte and plasma cell infiltration can also be seen. Hyperplasia occurs in the mucosa and submucosal nodules. Mucous glands hypertrophy, secretion hyperfunction, and increased mucus secretion occur. The disease is mainly improved by general treatment and drug therapy, the prognosis is poor, and the disease is prone to relapse. The patients are conscious of pharyngeal discomfort, dry, itchy, distension, secretions more and burning pain, easy to dry evil, sensation of foreign body which cannot come out or be swallowed; the above symptoms become a little more in talking, and aggravate after eating irritating food, fatigue or weather changes. Breathing and swallowing are unimpeded. Depending on the degree of the disease, it can be divided into chronic simple laryngitis, hypertrophic laryngitis and atrophic laryngitis.

The pathological changes of pharyngitis include chronic hyperemia of the pharyngeal mucosa, hyperplasia of the mucosa and submucosal connective tissue and lymphoid tissue, and formation of granular lymphoid follicles in the posterior pharyngeal wall. The pathological changes lead to the symptoms of sore throat, dry throat and foreign body sensation in the pharynx. The treatment principle of pharyngitis is to eliminate the cause, remove the local proliferation of lymphatic tissue, improve the body immunity, prevent acute upper respiratory tract infection. At present, although there are many clinical treatment methods, the effect is not ideal, since the fact of a large amount of medicine, taking the drug or aerosol for a long time causing larynx, trachea discomfortable, or spray cannot reach the lower pharynx, mostly employing the treatment of traditional Chinese medicine, or laser, microwave, freezing and so on. Simple drug treatment and long course of treatment are not easy to be accepted by patients. The mucosal recovery is relatively slow with local physiotherapy.

At present, the methods reported in the research papers and patent literature for the treatment of lung diseases are mainly drug treatment for mainly reducing cough and phlegm, and most of them are based on traditional Chinese medicine. The commonly used Chinese patent drugs are Tong Xuan Li Fei pills, comprising: perilla leaf, Peucedani Radix, platycodon, bitter almond, Ephedrae Herba, licorice, orange peel, pinellia (preparation), poria, fructus aurantii (stir-fried), scutellaria; Lian Hua Qing Wen capsules, comprising: forsythia, honeysuckle, stir-fried ephedra, stir-fried bitter almonds, Gypsum Fibrosum, Isatis root, Dryopteris Crassirhizoma, houttuynia, patchouli, rhubarb, Rhodiola rosea, menthol, licorice; Fang Feng Tong Sheng pills, comprising: ephedra, schizonepeta spike, parsnips, peppermint rhubarb, mirabilite, talc, Gardenia, Gypsum Fibrosum, scutellaria, forsythia, platycodon, angelica, paeoniae, chuanxiong, rhizoma atractylodes macrocephalae (fried), licorice; Ju Hong pills, comprising: Citri Grandis Exocarpium, pinellia (made), poria, licorice, platycodon, bitter almond, perilla seed (fried), aster, coltsfoot flower, pericarpium trichosanthis, Fritillaria thunbergii, rehmannia, ophiopogon, gypsum. Yang Yin Qing Fei pills, comprising: rehmannia, ophiopogon, scrophulariae, fritillaria, paeony, peony bark, mint, licorice, etc. Related research and prescriptions are composed of many traditional Chinese medicinal materials, including a variety of non-medicinal and edible homologous Chinese medicinal materials, and even toxic medicinal materials, the safety compatibility between drugs is not clear, quality standards are not easy to establish, the mechanism of drug action is not clear, most of the single day dose is larger (more than 50 g), taking a long time is easy to produce liver and kidney toxicity. Representative patents include such as patent CN111298011B for the treatment of pulmonary fibrosis, mainly comprising Astragalus membranaceus, rhodiola Rosea, scrophulariae, Fritillaria thunbergia, thule shell, raw oyster, dragon, silkworm, sea clam shell, waxgourd seed, chuanxiong, zedoary, peach kernel, platycodon, white mustard seed, draba seed; patent CN106924402B for the treatment of viral pneumonia mainly comprising: notopterygium root, rhizoma atractylodis, dandelion, scutellaria, raw astragalus, parsnip, houttuynia, trichosanthin; patent CN110870402B for the treatment of lung infection caused by 2019-nCoV mainly comprising: Ephedrae Herba, scorched licorice, almond, Raw gypsum, cassia twig, alissia, poria, white art, poria, bupleurum, scutellaria, ginger pinellia, ginger, aster, winter flower, shecan, asarum, yam, poncircirus aurantii, orange peel, patchouli. The above typical patents are characterized by a wide variety of medicinal materials, non-medicinal and edible homologous mostly thereof, and long-term use will have a certain impact on liver and kidney function.

After scientific compatibility research, the present application adopts the prescription of natural plants with the medicinal and edible homology, which has multiple functional mechanisms such as inhibiting the generation of inflammatory factors, regulating self-inflammatory response with non-antibiotic drugs, not involving antibiotic drug tolerance, clearing lung and moistens lung to protect lung, and quickly restoring lung function. Besides, for the patients with symptoms of pharyngitis such as dry pharyngeal pain, pharyngeal itching, cough, swollen throat, pharyngeal discomfort, it also has a therapeutic effect.

### SUMMARY

According to the clinical autopsy and pathology report, the early stage of the COVD-19, which originated at the end of 2019, attacked the human's lung, heart, liver, kidney, stomach and nervous system at the same time, causing multiple organ failure and damage. Especially after the virus infected the lungs, it directly triggered the lung inflammatory response, resulting in a large amount of tissue fluid oozing out and causing pulmonary edema. CT scans was examined with lung lesions such as ground glass shadows or veins, pulmonary fibrosis, effusion of alveoli, and emphysema. Common pulmonary sequelae include: pulmonary fibrosis, ground glass shadows, interstitial lung lesions, low oxygenation, and dyspnea. For patients with sequelae of COVID-19 infection, the prognosis of rehabilitation treatment is of paramount importance and should not be ignored.

The first purpose of the present application is to provide a medicinal and edible homologous traditional Chinese medicine composition for treating pulmonary sequelae or respiratory dysfunction of COVID-19 infected patients.

The second purpose of the present application is to provide a traditional Chinese medicine composition for the treatment of lung function abnormalities and pharyngitis caused by other influenza viruses, including but not limited to the COVID-19, in order to solve the existed insufficient side effects of the chemical drugs and proprietary Chinese medicines for the treatment of a series of symptoms caused by influenza virus, including the damaged bodily function.

The third purpose of the present application is to provide a preparation comprising a traditional Chinese medicine composition for the treatment of lung function abnormalities and pharyngitis caused by non-influenza virus.

The existing technology also discloses some traditional Chinese medicines for the treatment of pharyngitis, which are achieved by the compatibility principle of the medicinal materials of king and minister. The compatibility of traditional Chinese medicine can achieve certain effects, but the ingredients are complex, the daily dosage is relatively large, long-term administration is easy to produce liver and kidney toxicity, and the efficacy needs to be improved. Therefore, the present application provides the following technical solutions:

1. The traditional Chinese medicine composition is a main prescription comprising seven medicinal and edible homologous medicinal materials of monk fruit, cinnamon, prunella, houttuynia, platycodon, gardenia and licorice as active components. On the basis of the main prescription, the seven medicinal and edible homologous medicinal materials of tangerine (orange red), loquat leaf, angelica, burdock root, cloves, reed root and black pepper are added as the secondary prescription. The above formulation can be the formulation of the original medicinal materials, the formulation composed of the extractions of the active ingredients, or the formulation composed of the original medicinal materials and extractions thereof.

2. The composition of the Chinese medicine, characterized in that the formulation of the main prescription of composition can be the formulation comprising two or more in the group of main prescription; the formulation of the secondary prescription of composition can be the formulation comprising two or more in the group of secondary prescription.

The main prescription by weight comprising: 0-30 parts of monk fruit, 0-20 parts of cinnamon, 0-20 parts of prunella, 0-30 parts of houttuynia, 0.5-20 parts of platycodon, 0-20 parts of gardenia and 0.5-10 parts of licorice.

Preferably, the main prescription by weight comprising: 20-30 parts of monk fruit, 10-20 parts of cinnamon, 10-20 parts of prunella, 20-30 parts of houttuynia, 10-20 parts of platycodon, 10-20 parts of gardenia, and 5-10 parts of licorice.

The secondary prescription by weight comprising: 0.5-30 parts of tangerine (orange red), 0-30 parts of loquat leaf, 0.2-20 parts of angelica, 0-20 parts of burdock root, 0-10 parts of cloves, 0-20 parts of reed root, and 0-5 parts of black pepper.

Preferably, the secondary prescription by weight comprising: 20-30 parts of tangerine (orange red), 20-30 parts of loquat leaf, 10-20 parts of angelica, 10-20 parts of burdock root, 5-10 parts of cloves, 10-20 parts of reed root and 1-5 parts of black pepper.

The above composition is composed of the main prescription, or the main prescription and the secondary prescription mixed. The choice of the dosage of the medicinal materials or extracts equivalent to the medicinal materials for composition is affected by the weight of the subject and the degree of disease progression. In some embodiments, the total dosage may be approximately 0.001 to 20 g/kg body weight, approximately 0.005 to 10 g/kg body weight, approximately 0.01 to 5 g/kg body weight, approximately 0.01 to 2.5 g/kg body weight, approximately 0.02 to 1 g/kg body weight, approximately 0.02 to 0.3 g/kg body weight, approximately 0.02 to 0.2 g/kg body weight, approximately 0.02 to 0.1 g/kg body weight, or approximately 0.02 to 0.08 g/kg body weight. In some embodiments, the dosage is about 0.01 to 0.05 g/kg body weight. In another embodiment, the dosage is about 0.05 to 0.10 g/kg body weight.

3. The traditional Chinese medicine composition, characterized in that the active substances of each component are mainly flavonoids, saponins, polysaccharides, polyphenols and other components which are antibacterial, antiviral, anti-inflammatory, and can improve immunity and increase nutritional components. The total content range of the active ingredients of the formulation is as follows:

$$8. \% \leq Total Flavonoids \leq 26. \%$$

$$5. \% \leq Total polysaccharides \leq 20. \%$$

$$2. \% \leq Total Saponins \leq 16. \%$$

$$2. \% \leq Total polyphenols \leq 15. \%$$

4. The Chinese medicine composition can be made by combining the active substance extract of various single medicinal material, or by the active substance extract of the original medicinal materials mixed. The extract method of the active ingredient of any material, including the active ingredient of single medicinal material or medicinal materials mixed, may include but is not limited to the following steps:
(1) crushing the Chinese medicinal materials;
(2) soaking the herb powder with the extraction agent overnight (with or without the treatment of biological enzymes); wherein the extraction agent is water or ethanol, and when the extraction agent is ethanol, the ethanol concentration is 30%-95%;
(3) extracting the Chinese medicinal materials in step (2) by decocting or percolating at low speed;
(4) concentrating the extract to obtain the dried paste powder from the extracted liquid by rotary evaporation, spray drying or vacuum drying; or
the following steps:
(1) fully drying the medicinal materials of the formulation and then crushing them (30-80 mesh);
(2) putting the material obtained from step (1) into the extractor of supercritical equipment; the extraction medium is CO₂, the extraction temperature is 30-70° C., the extraction pressure is 5-60 MPa, and the extraction time is 1-4 hours to obtain the volatile oil extract;
(3) mixing proportionally the pharmaceutical residues collected in step (2) with the rest of the medicinal materials, and adding 8-12 folds of water, adjusting the pH value to 4-6, adding biological enzymes with an amount of 0.5-5% to the medicinal materials, wherein enzymatic hydrolysis is performed for 2-4 hours at 30-50° C., after enzymatic hydrolysis, performing the heating reflux extraction for enzymatic hydrolysis liquid for 60-180 mins and 1-3 times at 80-100° C.;
(4) combining directly the volatile oil extraction collected in step (2) with the extracted liquid obtained in step (3) to prepare the liquid dosage form or after combining directly, concentrating the extracted liquid and adding auxiliary materials to prepare the solid particles, or concentrating the extracted liquid, drying to obtain dry paste powder and adding auxiliary materials to prepare the solid dosage form.

5. The Chinese medicine composition can be prepared by combining the active substance extract of the single medicinal material or by the active substance extract of the original medicinal materials mixed, and the active ingredient can be obtained by supercritical extraction or separating the active substance group by macroporous resin.

6. The preparation method of the traditional Chinese medicine composition, characterized in that other nutritional components can be added as auxiliary materials of the preparation to make different dosage forms and enhance the above-mentioned effects. These effective auxiliary materials are nutritional supplements consisting of at least one or two or more of amino acids (glycine, arginine, leucine, valine, alanine, serine acid, proline, isoleucine, phenylalanine, lysine, threonine, methionine, tryptophan, histidine, tyrosine, aspartate, cysteine, glutamic acid), curcumine or turmeric extract, betaine hydrochloride, chlorogenic acid, reduced glutathione, yeast beta-glucan, vitamin C, gamma-aminobutyric acid, resveratrol, polyfructose, galacto-oligosaccharides, chitosan, sodium alginate.

7. The composition of Chinese medicine, characterized in that the preparation is a pharmaceutically acceptable oral preparation, including liquid preparation, substitute tea, granules, capsules, tablets, powder and other acceptable dosage forms.

8. The traditional Chinese medicine composition can be used to treat pulmonary disorders caused by influenza virus, including dyspnea, cough, pulmonary nodules, pulmonary fibrosis and other pulmonary diseases.

9. The Chinese medicine composition can be used for the treatment of pharyngeal diseases such as dryness and slight pain, pharyngeal itching and cough, sore and swollen throat, pharyngeal discomfort, etc.

10. The Chinese medicine composition can also be used for the treatment of other influenza viruses such as avian influenza virus, hand-foot-and-mouth disease, etc.

11. The Chinese medicine composition can also be used to treat respiratory tract related diseases such as lung grinding glass shadow, interstitial lung disease, COPD, etc.

12. The Chinese medicine composition can improve a series of symptoms such as abnormal lung function and pharyngitis caused by influenza virus or non-influenza virus, and avoid physical discomfort and side effects caused by other treatment means.

13. The above-mentioned Chinese medicine formulation for the treatment of lung function abnormalities and pharyngitis caused by influenza virus are characterized by the utilization of the characteristics of each component. Through the following mechanisms of antibacterial, anti-inflammatory, immune enhancement, endocrine improvement, cardiovascular function improvement, vitamin and other essential nutrients supplementation of the active ingredients group of Chinese medicine, achieve that the overall physical condition is improved and the lung function abnormalities caused by influenza virus, including respiratory dyspnea, cough, pulmonary fibrosis, pulmonary nodules, pulmonary glass shadow, interstitial pulmonary disease, COPD, pharyngitis and other respiratory diseases are alleviated and cured.

Monk fruit, with a sweet taste and cool nature, moistens the lungs to stop coughing and generates body fluids to quench thirst. It mainly contains cucurbitane triterpenoids, Siraitia grosvenorii polysaccharides, mannitol (including D-mannitol), proteins, amino acids, flavonoid glycosides, kaempferol-3,7-a-L-dirhamnoside and mogroside, etc. It has effects of antitussive, anti-asthmatic, antioxidant, anti-tumor, and blood glucose reduction.

Cinnamon, with a xin and sweet taste, and hot nature, tonifies fire to assist yang, guides fire back to its origin, disperses cold to relieve pain, and warms and unblocks the meridians. It contains volatile oil, tannins, mucilage, carbohydrates, etc. The main component of the oil is cinnamaldehyde at approximately 85% and cinnamyl acetate, along with small amounts of benzaldehyde, cinnamic acid, salicylic acid, benzoic acid, vanillin, phenylethyl acetate, etc. Cinnamaldehyde is the effective component responsible for Rou Gui's analgesic, sedative, and antipyretic actions.

Prunella, with a xin and bitter taste, and cold nature, clears heat and purges fire, brightens the eyes, and disperses nodules to reduce swelling. It mainly contains glycosides like prunella vulgaris polysaccharides, oleanolic acid, rutin, hyperoside, as well as organic acids like ursolic acid, caffeic acid, free oleanolic acid; it also contains vitamin B1, vitamin C, vitamin K, carotenes, resins, bitter substances, tannins, volatile oils, etc. It is suitable for lymphadenopathy, thyroid swelling, and has effects of clearing heat to disperse nodules, antibacterial, and anti-inflammatory.

Houttuynia, with a xin taste and cold-cool nature, enters the Lung channel. It can clear heat and detoxify, reduce swelling and treat boils, promote urination to remove dampness, stop dysentery due to heat, and strengthen the stomach to aid digestion. It contains volatile oils, including antibacterial components such as decanal, lauraldehyde, alpha-pinene, linalool, methyl n-nonanone, camphene, myrcene, limonene, bornyl acetate, eugenol. It also contains afzelin, hyperoside, rutin, chlorogenic acid, beta-sitosterol, stearic acid, oleic acid, linoleic acid. Modem pharmacological experiments have shown that it has effects of enhancing immunity, antibacterial, antiviral, diuretic, anti-tumor, and anti-inflammatory.

Platycodon, with a bitter and xin taste, and neutral nature, promotes lung function, benefits the throat, expels phlegm, and discharges pus. It contains Platycodon saponin aglycone, neooleandrin, platycodin A, B, C, platycodin aglycone-3-0-beta-glucoside, platycodin C, alpha-spinasterol, alpha-spinasterol-beta-D-glucoside, betulinol, Platycodon fructooligosaccharides, amino acids. It has expectorant and antitussive effects, hypoglycemic effects, anti-inflammatory effects, effects on the circulatory system, inhibition of gastric juice secretion, and anti-ulcer effects.

Gardenia, with a bitter taste and cold nature, purges fire to relieve irritability, clears heat and dries dampness, cools blood to detoxify. It mainly contains iridoid glycosides, polysaccharides, flavonoids, organic acids, coumarins, volatile oils, saponins, lignans, and various trace elements. It has effects of enhancing immunity, anti-inflammatory and bactericidal, diuretic, antioxidant, anti-tumor, antipyretic, and analgesic.

Licorice, with a sweet taste and neutral nature, tonifies the spleen to benefit qi, clears heat and detoxifies, expels phlegm and stops coughing, alleviates pain in emergencies, and harmonizes the actions of other herbs. It contains glycyrrhizin, glycyrrhetic acid, liquiritin, isoliquiritin, neoliquiritin, neoisoliquiritin, liquiritigenin, isoliquiritigenin, glabridin, glycyrrhizol, isoglycyrrhizol, 7-methylcoumarin, umbelliferone, and other active components. It has effects similar to adrenal corticosteroids, regulates immune function, treats ulcers, relieves spasms, protects the liver, soothes coughs, expels phlegm, reduces inflammation, counters allergic reactions, inhibits bacteria and viruses, detoxifies, corrects arrhythmias, lowers lipid levels, prevents arteriosclerosis, and combats tumors.

Tangerine (orange red), with a xin and bitter taste, and warm nature, regulates qi to widen the chest, dries dampness, and transforms phlegm. The fruit contains naringin, naringenin; the peel contains citral, geraniol, linalool, methyl anthranilate. It has effects on transforming phlegm and stopping coughs, treating chronic bronchitis, asthma, throat itching with excessive phlegm, chest tightness, vomiting, hiccups, food stagnation, indigestion from alcohol, long-term stomachache, and qi pain.

Loquat leaf, with a bitter and slightly xin taste, and slightly cold nature, clears the lungs to stop coughing, harmonizes the stomach to reduce upward rebellion, and quenches thirst. It contains amygdalin, ursolic acid, oleanolic acid, malic acid, citric acid, tannins, vitamin B1. It has antibacterial, analgesic, lung-clearing, phlegm-transforming, cough-stopping, and skin-nourishing effects, and is effective in preventing influenza, chronic bronchitis, hoarseness, and lung-heat-induced coughs.

Angelica, with a xin taste and warm nature, resolves exterior patterns to dispel cold, expels wind to relieve pain, unblocks nasal passages, reduces swelling, discharges pus, and relieves wind-induced itching. It mainly contains coumarins like oxypeucedanin, imperatorin, isoimperatorin, palmitic acid, stigmasterol, β-sitosterol, β-carotin, and trace elements such as Ca, Cu, Fe, Zn, Mn, Ni, Co, Cr, Mo. It has anti-inflammatory, antipyretic, analgesic, antispasmodic, hypotensive, antibacterial, photosensitive, anticancer, and anti-radiation effects.

Burdock root, with a bitter and slightly sweet taste, and cool nature, disperses wind-heat and detoxifies swellings. It mainly contains various essential amino acids, caffeic acid, chlorogenic acid, iso-chlorogenic acid, dicaffeoyl derivatives, inulin, and macro and microelements necessary for the human body such as Ca, Mg, Fe, Mn, Zn, which have diuretic, hypotensive, immunity-enhancing, anticancer, antimutagenic, growth-promoting, antibacterial, and antifungal effects.

Reed root, with a sweet taste and cold nature, clears heat and generates body fluids, alleviates irritability, stops vomiting, and promotes urination. It contains vitamins B1, B2, C, asparagine, amino acids, fatty acids, sterols, tocopherol, caffeic acid, gentisic acid, syringaldehyde, coniferaldehyde, vanillic acid, ferulic acid, coixol, trichosanthes saponin, 0-amyrin, taraxerol, taraxasterol, polysaccharides, proline, and betaines. It has antipyretic, sedative, analgesic, hypotensive, hypoglycemic, antioxidant, and estrogen-like effects, inhibits beta-hemolytic streptococcus, and the contained coixol inhibits skeletal muscle, while trichosanthin relaxes intestinal smooth muscle.

Cloves, with a xin taste and warm nature, warms the center to reverse downward qi flow, tonifies the kidneys to assist yang. It contains eugenol, acetyl eugenol, small amounts of α- and β-caryophyllene, humulene, piperitone, α-bisabolene, rhamnetin, naphthol, oleanolic acid, eugenin, eugenitin, isoeugenitin. It has anti-gastric ulcer, antidiarrheal, cholagogue, analgesic, anti-hypoxia, anticoagulant, antimutagenic, antibacterial, and insecticidal effects. It can alleviate abdominal distension, nausea, and vomiting and inhibit various pathogenic fungi, cocci, streptococci, pneumococci, dysentery bacilli, E. coli, typhoid bacilli, and influenza virus.

Black pepper, with a spicy and xin taste and hot nature, contains piperine, piperamide, pipernonaline, piperidine B, guineensine, pseudobulbine A, piperacid gum, etc. It has antibacterial, antioxidant, anticonvulsant, cholagogue, hypertensive, and anticancer effects.

14. During the present application, it was found that the active substance group contained in the medicinal herbs of prescription can eliminate inflammatory factors; improve immunity, improve lung function, improve intestinal function, supplement body nutrients and so on. It is embodied in: improving the body's immunity, promoting the proliferation and response of immune cells; improving lung function, increasing oxygenation capacity; improving bowel function and promoting intestinal motility.

Anti-inflammatory ingredients are as followed: oleanolic acid, ursolic acid, luteolin, kaempferol, 1,8-eucalyptus essential oil, geniposide, proanthocyanidin, ursolic acid, syringaldehyde, humulene, naringin, bergamolactone, hesperidin, glycyrrhetinic acid, glycyrrhizin, isoquercetin, rutin, hypericin, houttuynia, chlorogenic acid, myricetin, piperine, pepper essential oil, pepper oleoresin. The above active ingredients mainly exist in the medicinal materials of prescription of prunella, gardenia, cinnamon, loquat leaf, reed root, cloves, tangerine (orange red), licorice, angelica, houttuynia, black pepper and so on.

Ingredients enhancing immunity are as followed: oleanolic acid, catechin, glycyrrhetinic acid, hypericin, houttuynia. The above active ingredients mainly exist in the medicinal materials of prescription of prunella, cinnamon, loquat leaf, cloves, licorice, houttuynia and so on.

Ingredients improving lung function are as followed: podoside V, mannitol, luteolin, quercetin, kaempferol, β-sitosterol, amygdalin, alfalfa, platycodon D, polygallic acid, limonene, glycyrrhetinic acid, glycyrrhizic acid, oxypretin, europretin, angelicin. The above active ingredients mainly exist in the medicinal materials of prescription of monk fruit, prunella, loquat leaf, reed root, platycodon, tangerine (orange red), licorice, angelica, houttuynia etc.

Ingredients improving intestinal function are as followed: geniposide, cinnamaldehyde, alfalfa. The above active ingredients mainly exist in the medicinal materials of prescription of gardenia, cinnamon and other medicinal materials.

### Beneficial Effects

1. It is found that the main prescription of the present application: monk fruit, cinnamon, prunella, houttuynia, platycodon, gardenia and licorice can cooperate to improve the antiviral, antibacterial and anti-inflammatory ability of the body in many aspects, and the added secondary prescription: tangerine (orange red), loquat leaf, angelica, burdock root, cloves, reed root and black pepper can further improve the abnormal symptoms of the lung caused by influenza virus, including sore throat, dyspnea, persistent cough and other symptoms.

2, The study also found that the main prescription: monk fruit, cinnamon, prunella, houttuynia, platycodon, gardenia, licorice with the secondary prescription: tangerine (orange red), loquat leaf, angelica, burdock root, cloves, reed root, black pepper can further provide the body's defense ability, and have a better therapeutic effect on the symptoms of pharyngitis such as dry pharyngeal pain, pharyngeal itching and cough, sore throat, pharyngeal discomfort.

3. The initial purpose of this patent project is to develop a series of symptoms of pharyngitis. However, during the research, it was found that the formulation has good therapeutic effects on the sequelae of pulmonary dysfunction caused by influenza virus, especially the recently discovered COVID-19, including lung grinding glass shadow, interstitial lung disease, lung fibrosis, low oxygenation concentration and dyspnea.

4. The formulation of traditional Chinese medicine is based on the theory and experience of traditional Chinese medicine, and is compatible according to the methods of the king and the minister. Inspired by traditional Chinese medicine and traditional Chinese medicine prescriptions, the patent constructs the formulation with scientific and systematic thinking from the active group classification and pharmacological action mechanism of traditional Chinese medicine, and disease pathological mechanism, breaking the inherent compatibility concept of the king and the minister of the traditional formulation. At the same time, it integrates modern pharmaceutical technology to optimize the product process and dosage forms.

### DETAILED DESCRIPTION

Monk fruit (fructus momordicae): The dried fruit of Momordica grosvenori Swingle. (Purchased from Anhui Jincheng CO, Batch: 19102908)
Cinnamon (cortex cinnamomi): The dried tree barks of Cinnamomum cassia Presl. (Purchased from Anhui Jincheng CO, Batch: 19112001)
Prunella (spica prunellae): The dried fruit clusters of Prunella vulgaris L. (Purchased from Anhui Jincheng CO, Batch: 19080501)
Houttuynia (herba houttuyniae): The dried aboveground part of Houttuynia cordata Thunb. (Purchased from Anhui Jincheng CO, Batch: 19090102)
Platycodon (radix platycodi): The dried roots of Platycodon grandiflorum (Jacq.) A. DC. (Purchased from Anhui Jincheng CO, Batch: 19110601)
Gardenia (fructus gardeniae): The dried ripe fruit of Gardenia jasminoides Ellis. (Purchased from Hubei Jurui CO, Batch: 190201)
Licorice (radix glycyrrhizae): The dried roots of Glycyrrhiza uralensis Fisch., Glycyrrhiza inflata Bat., Glycyrrhiza glabra L. (Purchased from Anhui Jincheng CO, Batch: 190502)
Tangerine (orange red) (exocarpium citri rubrum): The dried outer pericarps of Citrus grandis Osbeck var. tomentosa Hort., Citrus reticulata Blanco or Citrus reticulata Blanco var. (Purchased from Anhui Jincheng CO, Batch: 200101)
Loquat leaf (folium eriobotryae): The dried leaves of Eriobotrya japonica (Thunb.) L. (Purchased from Hunan Zhenxing CO, Batch: 20020301)
Angelica (radix angelicae): The dried roots of Angelica dahurica (Fisch.ex Hoffm.) Benth. et Hook.f., Angelica dahurica (Fisch.ex Hoffm.) Benth.et Hook.f. var. formosana (Boiss.) Shan et Yuan. (Purchased from Anhui Jincheng CO, Batch: 191201)
Burdock root (radix bardanae): The roots of Arctium lappa L. (Purchased from Anhui Jincheng CO, Batch: 181101)
Cloves (flos caryophylli): The dried flower buds of Eugenia caryophllata Thunb. (Purchased from Anhui Jincheng CO, Batch: 180701)
Reed root (rhizoma phragmitis): The fresh or dried roots of Phragmites communis Trin. (Purchased from Anhui Jincheng CO, Batch: 191101)
Black pepper (fructus piperis nigri): The dried ripe or nearly matured fruit of Piper nigrum L. (Purchased from Anhui Tengxin CO, Batch: 181101) The number of parts in the following examples refers to the number of parts by weight.

### Example 1

The following fully dried materials were weighed and taken: 30 parts of monk fruit, 20 parts of cinnamon, 20 parts of prunella, 30 parts of houttuynia, 20 parts of platycodon, 20 parts of gardenia, 10 parts of licorice, 30 parts of tangerine (orange red), 30 parts of loquat leaf, 20 parts of angelica, 20 parts of burdock root, 10 parts of cloves, 20 parts of reed root and 5 parts of black pepper, the above materials were raw or processed medicinal materials; then superfinely crushed and mixed, bagged or made into pills with honey. They were bagged and stored in the dry environment.

### Example 2

The following fully dried materials were weighed and taken: 20 parts of monk fruit, 10 parts of cinnamon, 10 parts of prunella, 20 parts of houttuynia, 10 parts of platycodon, 10 parts of gardenia, 5 parts of licorice, 20 parts of tangerine (orange red), 20 parts of loquat leaf, 10 parts of angelica, 10 parts of burdock root, 5 parts of cloves, 10 parts of reed root and 2 parts of black pepper, the above materials were raw or processed medicinal materials; then superfinely crushed; added with water in 10 folds of total weight of the materials for soaking overnight; decocted twice for 2 hours each time, filtrated; and the filtrate was evaporated at 70° C. to remove the water under reduced pressure and a concentrated liquid with a density of 1.1-1.2 was obtained, and the liquid is bottled or made into oral liquid.

### Example 3

The following fully dried materials were weighed and taken: 30 parts of monk fruit, 10 parts of cinnamon, 20 parts of prunella, 20 parts of houttuynia, 10 parts of platycodon, 10 parts of gardenia and 5 parts of licorice, then superfinely crushed; added with water in 10 folds of total weight of the materials was added for soaking overnight; decocted twice for 2 hours each time, filtrated and the filtrate was evaporated at 70° C. to remove the water under reduced pressure and a concentrated liquid with a density of 1.1-1.2 was obtained, and the liquid was bottled or made into oral liquid or fully concentrated and dried into powder.

The following fully dried materials were weighed and taken: 20 parts of tangerine (orange red), 30 parts of loquat leaf, 10 parts of angelica, 10 parts of burdock root, 10 parts of cloves, 20 parts of reed root and 2 parts of black pepper the above materials were raw or processed medicinal materials; then superfinely crushed; added with water in 10 folds of total weight of the materials was added for soaking overnight; decocted twice for 2 hours each time, filtrated and the filtrate was evaporated at 70° C. to remove the water under reduced pressure and a concentrated liquid with a density of 1.1-1.2 was obtained, and the liquid was bottled or made into oral liquid or fully concentrated and dried into powder.

The two extracts obtained above were mixed, further processed and prepared into tablets, capsules, granules and oral liquid, etc.

### Example 4

The following fully dried materials, raw or processed medicinal materials, were superfinely crushed respectively: monk fruit, cinnamon, prunella, houttuynia, platycodon, gardenia, licorice, tangerine (orange red), loquat leaf, angelica, burdock root, cloves, reed root and black pepper.

The crushed medicinal materials of monk fruit were taken, added with 10 folds of water and heated and extracted twice for 2 hours each time at 90° C., and then extracted liquid was concentrated to a certain density and spray-dried to obtain the dry powder.

The crushed medicinal materials of cinnamon were taken, and put into the extractor of supercritical equipment wherein the extraction medium was CO₂, the extraction temperature was 50° C., the extraction pressure was 45 MPa, and the extraction time was 3 hours, and the extract of volatile oil was obtained. The medicinal residue was added with water in 8 folds of the medicinal materials and heated and extracted twice for 2 hours each time at 90° C. Then the filtrate was combined and evaporated at 70° C. to remove the water under reduced pressure and spray-dried to obtain the dry powder.

The crushed medicinal materials of prunella were taken, added with 15 folds of water and heated and extracted twice for 2 hours each time at 90° C. Then the extracted liquid was added with 2 folds of 95% ethanol and the alcohol-precipitated liquid was concentrated to 1.07-1.12 (30° C.) and spray-dried to obtain the dry powder.

The crushed medicinal materials of houttuynia were taken, and put into the extractor of supercritical equipment wherein the extraction medium was CO₂, the extraction temperature was 45° C., the extraction pressure was 30 MPa, and the extraction time was 1.5 hours, and the extract of volatile oil was obtained. The medicinal residue was added with water in 10 folds of the medicinal materials and heated and extracted twice with 2 hours each time. Then the filtrate was combined and concentrated to a certain concentration at 70° C. under reduced pressure and spray-dried to obtain the dry powder.

The crushed medicinal materials of platycodon were taken, added with water at 60° C. in 10 folds of the medicinal materials for soaking 2 hours, extracted by heating reflux twice for 2 hours each time at 80° C. Then the extracted liquid was concentrated to a certain concentration and spray-dried to obtain the dry powder.

The crushed medicinal materials of gardenia were taken, and put into the extractor of supercritical equipment wherein the extraction medium was CO₂, the extraction temperature was 46° C., the extraction pressure was 34 MPa, and the extraction time was 2 hours, and the extract of volatile oil was obtained. Then the medicinal residue was added with 10 folds of the water and extracted by heating reflux twice for 2 hours each time. Then the filtrate was combined and concentrated to a certain concentration and spray-dried to obtain the dry powder.

The crushed medicinal materials of licorice were taken, added with 10 folds of water and heated and extracted twice for 2 hours each time at 100° C. Then the filtrate was combined and concentrated to a certain density and spray-dried to obtain the dry powder.

The crushed medicinal materials of tangerine (orange red) were taken, added with 10 folds of water and heated and extracted twice for 2 hours each time at 90° C. The extracted liquid was added with 95% ethanol to a concentration of 70%. Then the alcohol-precipitated liquid was concentrated to 1.07-1.12 (30° C.) and spray-dried to obtain the dry powder.

The crushed medicinal materials of loquat leaf were taken, added with 10 folds of 75% ethanol and extracted by heating reflux twice for 2 hours each time. Then the extract liquid was filtrated, combined and concentrated to a certain density and spray-dried to obtain the dry powder.

The crushed medicinal materials of angelica were taken, added with 8 folds of 75% ethanol and extracted by heating reflux thrice for 3 hours each time. Then the extract liquid was filtrated, combined and concentrated to a certain density and spray-dried to obtain the dry powder.

The crushed medicinal materials of burdock root were taken, added with 15 folds of water and extracted by heating reflux thrice for 3 hours each time at 95° C. Then the extract liquid was filtrated, combined and concentrated to a certain density and spray-dried to obtain the dry powder.

The crushed medicinal materials of reed root were taken, added with 0.5% biological enzyme and adjusted the pH to 4.5 wherein the enzymatic hydrolysis temperature was 40° C., the enzymatic hydrolysis time was 2 hours, soaked in the thermostatic water bath for extraction. Then the enzyme-extracted liquid was inactivated by heating for 2 minutes at high temperature and filtrated. Then the filtrate was concentrated to a certain concentration and spray-dried to obtain the dry powder.

The crushed medicinal materials of cloves were taken, and put into the extractor of supercritical equipment wherein the extraction medium was CO₂, the extraction temperature was 40° C., the extraction pressure was 30 MPa, and the extraction time was 2.5 hours, and the extract of volatile oil was obtained. The medicinal residue was added with 10 folds of the water and extracted by heating reflux twice for 2 hours each time. Then the filtrate was combined and concentrated to a certain concentration and spray-dried to obtain the dry powder.

The crushed medicinal materials of black pepper were taken, added with 2.5% biological enzyme and adjusted the pH to 5.0 wherein the enzymolysis temperature was 50° C. and the enzymolysis time was 4 hours, added with the 90% ethanol in 10 folds of the amount of medicinal material, soaked and extracted for 2 hours. Then the filtrate was concentrated to a certain concentration and spray-dried to obtain the dry powder.

The above dried powder of the medicinal materials were mixed and packaged as followed: 10 parts of monk fruit, 5 parts of cinnamon, 5 parts of prunella, 10 parts of houttuynia, 2 parts of platycodon, 5 parts of gardenia, 2 parts of licorice, 10 parts of tangerine (orange red), 10 parts of loquat leaf, 2 parts of angelica, 5 parts of burdock root, 2 parts of cloves, 5 parts of reed root and 0.5 parts of black pepper, or packaged by adding excipients for preparing different dosage forms.

### Example 5

The dried powders of extraction in example 4 were taken with or without the corresponding ultra-fine powder of raw medicinal materials and mixed equably, bagged or made into pills, then packaged.

### Example 6

The following fully dried materials were weighed and taken: 30 parts of monk fruit, 20 parts of cinnamon, 20 parts of prunella, 30 parts of houttuynia, 20 parts of platycodon, 20 parts of gardenia, 10 parts of licorice, 30 parts of tangerine (orange red), 30 parts of loquat leaf, 20 parts of angelica, 20 parts of burdock root, 10 parts of cloves, 20 parts of reed root and 5 parts of black pepper, the above materials were raw or processed medicinal materials; then added with the specific microorganisms (Aspergillus oryzae, Monascus, yeast, lactic acid bacteria, Bacillus, subtilis) separately for treating with microbial fermentation. Or the above medicinal materials were weighed and mixed, added with above microorganisms for treating with microbial fermentation. The post-treated medicinal materials were sterilized, added with water in 10 folds of the total weight of the medicinal materials, soaked overnight, decocted twice for 2 hours each time, and filtrated. The filtrate was evaporated at 70° C. to remove the water under reduced pressure to obtain a concentrated liquid with a density of 1.1-1.2. The concentrated liquid was bottled or made into oral liquid. Or the concentrated liquid of single medicinal material was spay-dried to obtain the dry powder directly with or without the corresponding ultra-fine powder of raw medicinal materials, and mixed equably, bagged or made into pills, packaged. Or the concentrated liquid of single medicinal material was spay-dried to obtain the dry powder directly, weighed according to the above proportion, and packaged by adding excipients for preparing different dosage forms.

### Example 7

The following fully dried materials were weighed and taken: 30 parts of monk fruit, 20 parts of cinnamon, 20 parts of prunella, 30 parts of houttuynia, 20 parts of platycodon, 20 parts of gardenia, 10 parts of licorice, 30 parts of tangerine (orange red), 30 parts of loquat leaf, 20 parts of angelica, 20 parts of burdock root, 10 parts of cloves, 20 parts of reed root and 5 parts of black pepper, the above materials were raw or processed medicinal materials; then added with the specific biological enzymes (cellulase, pectinase, lignin enzyme) separately for enzymatic treating. Or the above medicinal materials were weighed and mixed, added with above biological enzymes for enzymatic treating. The post-treated medicinal materials were sterilized, added with water in 10 folds of the total weight of the medicinal materials, soaked overnight, decocted twice for 2 hours each time, and filtrated. The filtrate was evaporated at 70° C. to remove the water under reduced pressure to obtain a concentrated liquid with a density of 1.1-1.2. The concentrated liquid was bottled or made into oral liquid. Or the concentrated liquid of single medicinal material was spay-dried to obtain the dry powder directly with or without the corresponding ultra-fine powder of raw medicinal materials, and mixed equably, and bagged or made into pills, packaged. Or the concentrated liquid of single medicinal material was spay-dried to obtain the dry powder directly, weighed according to the above proportion, and packaged by adding excipients for preparing different dosage forms.

### Example 8

In the above examples, the dried powder of extraction or the ultra-fine powder of raw medicinal materials can also be added with different excipients to make different dosage forms. The excipients added and dosage forms were as followed:

The ultra-fine powder of raw medicinal materials was mixed with silica evenly and bagged, or added with water, honey or other adhesives for making pills.

The dried powder of medicinal materials extraction was mixed, added with the nutritional supplement for example betaine hydrochloride, chlorogenic acid, yeast β-glucan and γ-aminobutyric acid, resveratrol, polyfructose, galacto-oligosaccharides, chitosan, sodium alginate; added with honey, isomalt oligosaccharide powder and powdered sugar for making oral liquid or paste.

The dried powder of medicinal materials extraction was mixed, added with the nutritional supplement for example curcumin or turmeric extract, amino acid, betaine hydrochloride, chlorogenic acid, reduced glutathione, vitamin B6, B2, gamma-aminobutyric acid, resveratrol, chitosan, sodium alginate, aloe vera gel of curacao;

The excipients for preparation were added: dextrin, starch, sucrose, lactose, microcrystalline cellulose, mannose, xylitol, bifidose and trehalose were selected as fillers.

Carboxymethyl cellulose sodium, povidone, sodium alginate and polyethylene glycol were selected as adhesives.

Dried starch, sodium carboxymethyl starch, crosslinked polyethylpyrrolidone, sodium crosslinked carboxymethyl cellulose and low substituted hydroxypropyl cellulose were selected as disintegrators.

Magnesium stearate, micro-powder silica gel, talc powder and polyethylene glycol were selected as lubricants.

Water, ethanol of different concentrations, or a mixture thereof were selected as wetting agent.

The above raw and auxiliary materials were mixed for making granule, tablet or capsule dosage forms.

The dosage forms in this patent are not limited to that mentioned in the several examples, but also include various clinically acceptable dosage forms.

### Example 9

The following dried powders of extraction were weighted and taken: 20 parts of monk fruit extraction powder, 10 parts of cinnamon, 10 parts of prunella, 20 parts of houttuynia, 10 parts of platycodon, 10 parts of gardenia, 5 parts of licorice, 20 parts of tangerine (orange red), 20 parts of loquat leaf, 10 parts of angelica, 10 parts of burdock root, 5 parts of cloves, 10 parts of reed root and 1 part of black pepper, mixed together, added with 0.5 part of betaine hydrochloride, 0.2 part of resveratrol, 0.3 part of galacto-oligosaccharides and 0.1 part of silica, and added with honey for making pills, bagged and stored in dry environment.

### Example 10

The following dried powders of extraction were weighed and taken: 20 parts of monk fruit, 20 parts of cinnamon, 20 parts of prunella, 20 parts of houttuynia, 20 parts of platycodon, 20 parts of gardenia, 20 parts of licorice, 20 parts of tangerine (orange red), 20 parts of loquat leaf, 20 parts of angelica, 20 parts of burdock root, 20 parts of cloves, 20 parts of reed root and 20 parts of black pepper, mixed, then added with ultra-fine powder of raw medicinal materials in one third of the weight of the respective medicinal materials according to the above formulation proportion of monk fruit, cinnamon, prunella, houttuynia, platycodon, gardenia, licorice, tangerine (orange red), loquat leaf, angelica, burdock root, cloves, reed root and black pepper, and added with 0.3 part of betaine hydrochloride, 0.2 part of resveratrol and 0.3 part of galacto-oligosaccharides bagged, or added with honey for making pills, bagged and stored in dry environment.

### Example 11

The following dried powders of extraction were weighted and taken: 20 parts of monk fruit, 20 parts of cinnamon, 10 parts of prunella, 20 parts of houttuynia, 10 parts of platycodon, 10 parts of gardenia and 5 parts of licorice, mixed, then added with ultra-fine powder of raw medicinal materials in one third of the weight of the respective medicinal materials according to the above formulation proportion of monk fruit, cinnamon, prunella, houttuynia, platycodon, gardenia and licorice, and added with 0.2 part of betaine hydrochloride, 0.1 part of chlorogenic acid, 0.3 part of galacto-oligosaccharides and 0.2 part of sodium alginate, bagged, or added with honey for making pills, bagged and stored in dry environment.

The following dried powders of extraction were weighed and taken: 20 parts of tangerine (orange red), 20 parts of loquat leaf, 10 parts of angelica, 10 parts of burdock root, 10 parts of cloves, 10 parts of reed root and 5 parts of black pepper, mixed together, then added with ultra-fine powder of raw medicinal materials in one third of the weight of the respective medicinal materials according to the above formulation proportion of tangerine (orange red), loquat leaf, angelica, burdock root, cloves, reed root and black pepper, and added with 0.2 part of betaine hydrochloride, 0.1 part of chlorogenic acid, 0.1 part of resveratrol and 0.3 part of polyfructose, bagged, or added with honey for making pills, bagged and stored in dry environment.

The two mixtures obtained above can be further processed and prepared into tablets, capsules, granules or oral liquids, etc.

### Example 12

The following dried powders of extraction were weighed and taken: 10 parts of monk fruit, 8 parts of cinnamon, 8 parts of prunella, 10 parts of houttuynia, 8 parts of platycodon, 8 parts of gardenia, 5 parts of licorice, 10 parts of tangerine (orange red), 10 parts of loquat leaf, 8 parts of angelica, 8 parts of burdock root, 5 parts of cloves, 8 parts of reed root and 2 parts of black pepper, added with 0.1 part of histidine, 0.1 part of curcumin, 0.2 part of chlorogenic acid, 0.2 part of polyfructose, 0.1 part of galacto-oligosaccharides, added with povidone K30, sodium carboxymethyl cellulose and sodium carboxymethyl starch, and mixed equably for preparing granules with 90% ethanol, then dried, tableted and bagged or bottled for storage.

### Example 13

The following fully dried materials were weighed and taken: 30 parts of monk fruit, 20 parts of cinnamon, 20 parts of prunella, 30 parts of houttuynia, 20 parts of platycodon, 20 parts of gardenia, 10 parts of licorice, 30 parts of tangerine (orange red), 30 parts of loquat leaf, 20 parts of angelica, 20 parts of burdock root, 10 parts of cloves, 20 parts of reed root and 5 parts of black pepper, and the above materials were raw or processed medicinal materials; then superfinely crushed; added with water in 10 folds of total weight of the materials for soaking overnight; decocted twice for 2 hours each time, filtrated. The filtrate was evaporated at 70° C. to remove the water under reduced pressure and a concentrated liquid with a density of 1.1-1.2 was obtained and dried to obtain the dried paste powder. The dried paste powder was added with 0.1 part of leucine, 0.1 part of curcumin, 0.1 part of chlorogenic acid, 0.1 part of resveratrol, 0.1 part of gamma-aminobutyric acid, 0.1 part of galacto-oligosaccharides, added with povidone K30, sodium carboxymethyl cellulose and sodium carboxymethyl starch, mixed equably for preparing granules with 90% ethanol, then dried, filled capsule or tableted, and bagged or bottled for storage.

### Example 14

The following fully dried materials were weighed and taken: 30 parts of monk fruit, 20 parts of cinnamon, 20 parts of prunellae, 30 parts of houttuynia, 20 parts of platycodon, 20 parts of gardenia and 10 parts of licorice, and the above materials were raw or processed medicinal materials; then superfinely crushed; added with water in 10 folds of the total weight of the materials for soaking overnight; decocted twice for 2 hours each time; filtrated. The filtrate was evaporated at 70° C. to remove the water under reduced pressure and a concentrated liquid with a density of 1.1-1.2 was obtained and dried to obtain the dried paste powder. The dried paste powder was added with 0.1 part of lysine, 0.1 part of chlorogenic acid, 0.01 part of vitamin B6, 0.1 part of reduced glutathione, 0.1 part of galacto-oligosaccharides, added with povinone K30, sodium carboxymethyl cellulose and sodium carboxymethyl starch, mixed equably for preparing granules with 90% ethanol, then dried, tableted, and bagged or bottled for storage.

The following fully dried materials were weighed and taken: 30 parts of tangerine (orange red), 30 parts of loquat leaf, 20 parts of angelica, 20 parts of burdock root, 10 parts of cloves, 20 parts of reed root and 5 parts of black pepper, and the above materials were raw or processed medicinal materials; then superfinely crushed; added with water in 10 folds of the total weight of the materials for soaking overnight; decocted twice for 2 hours each time; filtrated. The filtrate was evaporated at 70° C. to remove the water under reduced pressure and a concentrated liquid with a density of 1.1-1.2 was obtained and dried to obtain dried paste powder. The dried paste powder was added with 0.1 part of L-tyrosine, 0.1 part of curcumin, 0.1 part of chlorogenic acid, 0.1 part of resveratrol, 0.1 part of polyfructose, 0.1 part of sodium alginate, added with povinone K30, sodium carboxymethyl cellulose, sodium carboxymethyl starch and crosslinked povinone, mixed evenly for preparing granules by dry type granulator, then dried, tableted, and bagged or bottled for storage.

The two mixtures obtained above can be further processed and prepared into tablets, capsules, granules or oral liquid, etc.

### Clinical Cases Citated

The information of patients who were administered with the research products of this project are classified and sorted as follows:

The following information was collected: there were 68 patients aged between 24 and 70 years, and 28 cases among them were patients who had been discharged from hospital after recovering from COVID-19 with negative conversation. The above patients separately took the products in example 13 at different periods from 2020.4 to 2021.10, and took 10 g each time, three times a day in warm water. The administration cycle of this product ranged from one week to three months. No other drugs were taken during the use of this product.

### Typical Clinically Symptoms:

Pulmonary ground-glass opacity: ground-glass opacity in the lungs by CT examination;
Pulmonary fibrosis: dry cough, progressive dyspnea, obviously in post-activity;
COPD: chronic cough, cough of phlegm, shortness of breath, dyspnea;
Pharyngitis: dry pharynx, burning, itching, pharyngeal foreign body sensation, obstructive sensation.

### Criteria for Curative Effect:

Cure: all discomfort symptoms disappeared, and the relevant detection indexes were normal.
Effective: all discomfortable symptoms disappeared, and the relevant detection indicators were slightly deviated.

Ineffective: no significant improvement in clinical symptoms.

Treatment effect:

| | | | | | Total |
|---|---|---|---|---|---|
| | | | | | effective |
| Groups | Cases | Cure(%) | Effective(%) | Ineffective(%) | rate(%) |
| Pulmonary | 15 | 9(60.0) | 5(33.3) | 1(6.7) | 93.3 |
| ground- | | | | | |
| glass opacity | | | | | |
| Pulmonary | 13 | 8(61.5) | 4(30.8) | 1(7.7) | 92.3 |
| fibrosis | | | | | |
| COPD | 18 | 12(66.7) | 4(22.2) | 2(11.1) | 88.9 |
| Pharyngitis | 22 | 15(68.2) | 5(22.7) | 2(9.1) | 90.9 |

Conclusion: All patients in each group took the drug for a period of time, the discomfortable symptoms were disappeared, or significantly improved.

### Classic Case Report

Case 1 Mr. Xu, 59 Years Old

On January 20, he started to have a fever and took medicine in isolation at home. On January 23, his symptoms worsened with dry cough, wheezing, and chest pain. A CT scan of his chest showed a slight grinding glass shadow, which was confirmed as COVID-19 infection. The granules in example 13 on January 25 were taken, 10 g each time, three times a day in warm water within 6 consecutive days. Fever began to bring down on February 1, phlegm had been present in throat before taking, but phlegm disappeared after taking, and all discomfortable symptoms disappeared.

### Case 2 Xin, 46 Years Old

A low-grade fever was reported from February 18 for 9 consecutive days, accompanied with a cough for more than 10 days, and symptoms of dyspnea, low-grade fever, cough, dyspnea, and loss of appetite. CT scan of his lungs showed ground glass shadows and confirmed COVID-19 infection. On March 2, the granules in example 13 were taken, 10 g each time, three times a day in warm water. After 4 days of taking granules, low-grade fever, cough, dyspnea and other symptoms disappeared. After 10 days of taking granules was continued, CT scan of his lungs showed that the ground glass shadow disappeared, all discomfortable symptoms disappeared, now he obviously felt much better than before in illness and the immunity significantly improved.

Case 3: Ms. He, 38 Years Old

COVID-19 pneumonia was diagnosed on February 10, accompanied with symptoms of low-grade fever, sore throat, and weakness of limbs. After hospitalization, the symptoms were worsened and the lungs showed the ground glass shadow. Subsequently, she was recommended to take the granules in example 13 with 10 g each time, three times a day in warm water, and the discomfortable symptoms were improved after continuously using for a week. Continued to take over 10 days, the ground glass shadow disappeared and all the discomfortable symptoms disappeared.

Case 4 Mr. Wu, 56 Years Old Self-described as a patient with chronic pharyngitis, accompanied with severe laryngeal swelling and pain, throat itching and cough, and pharyngeal discomfort. Taking a variety of drugs as treatment of pharyngitis weren't effective, in early March the granules in example 13 were taken with 10 g each time, three times a day in warm water. After one week of continuous granules taking, laryngeal discomfort relieved, and after one week of taking granules was continued, throat discomfort disappeared without repeatedly.
Case 5 Ms. Xian, 30 Years Old

Due to frequently professional training for employees recently, the symptoms of sore throat, foreign body sensation and hoarse voice appeared. The granules product in example 13 were taken with 10 g each time, three times a day in warm water for a week, and the foreign body sensation in the throat disappeared, throat discomfort symptoms disappeared during the training, the voice returned to normal. Then one week of taking granules was continued for consolidating, the discomfort symptoms did not appear again.

Case 6 Mr. Wang, 68 Years Old

When he was young, he engaged in construction work and was diagnosed with COPD for 7 years. Climbing buildings or doing physical work would cause symptoms of dyspnea, which mainly manifested as dyspnea and increased sputum volume. A history of smoking for 26 years, spirometer FEV1:51% pred. The granules in example 13 were taken with 10 g each time, three times a day in warm water. After 10 days, dyspnea symptoms were relieved, sputum volume was reduced, and dyspnea symptoms disappeared after continued using for one month, basically no symptoms of expectoration were observed. Spirometer FEV1:78% pred. Then continuously using products for two months for consolidating, discomfort symptoms did not appear again.

It should also be emphasized that the above description is only the preferred cases of the present application and is not used to limit the present application. For the technical one in the field, increasing or reducing the ingredients or steps thereof according to the embodiments provided by the present application, or applying the present application to other technical fields closed to the present application are within the scope of protection of the present application.

1. A traditional Chinese medicine composition, characterized in that the traditional Chinese medicine composition comprising the main prescription with seven medicinal and edible homologous medicinal herbs of monk fruit, cinnamon, prunella, houttuynia, platycodon, gardenia and licorice as active components, and the main prescription is a formulation comprising two or more of the seven medicinal and edible homologous medicinal herbs of the main prescription;
optionally, on the basis of the main prescription, supplement seven medicinal and edible homologous medicinal herbs of tangerine (orange red), loquat leaf, angelica, burdock root, cloves, reed root, black pepper as the secondary prescription, the secondary prescription is a formulation comprising two or more of seven medicinal and edible homologous medicinal herbs of the secondary prescription;
optionally, the above prescription is the formulation of the original medicinal material, the formulation composed of its active ingredient extract, or the formulation composed of the original medicinal material and the active ingredient extract thereof.

2. The Chinese medicine composition claimed in claim 1, characterized in that the main prescription of the composition is a formulation of two or more of the seven medicinal and edible homologous medicinal herbs of the main prescription, and the secondary prescription of the composition is a formulation two or more of the seven medicinal and edible homologous medicinal herbs of the secondary prescription;
the main prescription by weight comprising: 0-30 parts of monk fruit, 0-20 parts of cinnamon, 0-20 parts of prunella, 0-30 parts of houttuynia, 0.5-20 parts of platycodon, 0-20 parts of gardenia, and 0.5-10 parts of licorice;
preferably, the main prescription by weight comprising: 20-30 parts of monk fruit, 10-20 parts of cinnamon, 10-20 parts of prunellae, 20-30 parts of houttuynia, 10-20 parts of platycodon, 10-20 parts of gardenia, 5-10 parts of licorice;
the secondary prescription by weight comprising: 0.5-30 parts of tangerine (orange red), 0-30 parts of loquat leaf, 0.2-20 parts of angelica, 0-20 parts of burdock root, 0-10 parts of cloves, 0-20 parts of reed root, and 0-5 parts of black pepper;
preferably, the secondary prescription by weight comprising: 20-30 parts of tangerine (orange red), 20-30 parts of loquat leaf, 10-20 parts of angelica, 10-20 parts of burdock root, 5-10 parts of cloves, 10-20 parts of reed root, and 1-5 parts of black pepper;
the above composition is composed of the main prescription, or the main prescription and the secondary prescription mixed; the dosage of the medicinal materials or extracts equivalent to the medicinal materials for composition; preferably, the total dosage can be approximately 0.001 to 20 g/kg of subject body weight, approximately 0.005 to 10 g/kg of subject body weight, approximately 0.01 to 5 g/kg of subject body weight, approximately 0.01 to 2.5 g/kg of subject body weight, approximately 0.02 to 1 g/kg of subject body weight, approximately 0.02 to 0.3 g/kg of subject body weight, approximately 0.02 to 0.2 g/kg of subject body weight, approximately 0.02 to 0.1 g/kg of subject body weight, or approximately 0.02 to 0.08 g/kg of subject body weight; or, preferably, the dosage is about 0.01 to 0.05 g/kg of subject body weight; Or, preferably, the dosage is approximately 0.05 to 0.10 g/kg of subject body weight.

3. The Chinese medicine composition claimed in claim 1, characterized in that the active substances of each component are flavonoids, saponins, polysaccharides and polyphenols, and the total content range of the active ingredients of the formulation is as follows:
$$8. \% \leq total flavonoids \leq 26. \%$$
$$5. \% \leq total polysaccharides \leq 20. \%$$
$$2. \% \leq total saponins \leq 16. \%$$
$$2. \% \leq total polyphenols \leq 15. \% .$$

4. The Chinese medicine composition claimed in claim 1, characterized in that the Chinese medicine composition is a pharmacologically acceptable oral preparation, including liquid preparation, substitute tea, granule, capsule, tablet, powder and other acceptable dosage forms.

5. The method for preparing the Chinese medicine composition claimed in claim 1, characterized in that the Chinese medicine composition can be prepared by combining the active substance extracts of various single medicinal material, or by combining the active substance extracts of the original medicinal materials mixed;
the method of extracting the active ingredients of any medicinal material may include, but is not limited to, the following steps:
(1) crushing the Chinese medicinal materials;
(2) soaking the herb powder with the extraction agent overnight (with or without the treatment of biological enzymes) wherein the extraction agent is water or ethanol, and when the extraction agent is ethanol, the ethanol concentration is 30%-95%;
(3) extracting the Chinese medicinal materials in step (2) by decocting or percolating at low speed;
(4) concentrating the extract to obtain the dried paste powder from the extracted liquid by rotary evaporation, spray drying or vacuum drying;
or the following steps:
(1) fully drying the medicinal materials of the formulation and then crushing them (30-80 mesh);
(2) putting the material obtained from step (1) into the extractor of supercritical equipment; the extraction medium is CO₂, the extraction temperature is 30-70° C., the extraction pressure is 5-60 MPa, and the extraction time is 1-4 hours to obtain the volatile oil extract;
(3) mixing proportionally the pharmaceutical residues collected in step (2) with the rest of the medicinal materials, and adding 8-12 folds of water, adjusting the pH value to 4-6, adding biological enzymes with an amount of 0.5-5% to the medicinal materials, wherein enzymatic hydrolysis is performed for 2-4 hours at 30-50° C., after enzymatic hydrolysis, performing the heating reflux extraction for enzymatic hydrolysis liquid for 60-180 mins and 1-3 times at 80-100° C.;
(4) combining directly the volatile oil extract collected in step (2) with the extracted liquid obtained in step (3) to prepare the liquid dosage form, or
after combining directly, concentrating the extracted liquid and adding auxiliary material to prepare solid particles, or
concentrating the extracted liquid, drying to obtain dry paste powder and adding auxiliary material to prepare the solid dosage form.

6. The method claimed in claim 5, characterized in that the Chinese medicine composition can be prepared by combining the active substance extract of the single medicinal material or the active substance extract of the original medicinal materials mixed, wherein the extraction of the active substance is obtained by separating the active substance by supercritical extraction or by macroporous resin.

7. The method claimed in claim 5, characterized in that other nutrients can be added as auxiliary materials of the preparation to prepare different dosage forms for enhancing the effect of the above-mentioned preparation; these effective auxiliary materials are nutritional supplements consisting of at least one or two of amino acids (glycine, arginine, leucine, valine, alanine, serine acid, proline, isoleucine, phenylalanine, lysine, threonine, methionine, tryptophan, histidine, tyrosine, aspartate, cysteine, glutamic acid), curcumine or turmeric extract, betaine hydrochloride, chlorogenic acid, reduced glutathione, yeast beta-glucan, vitamin C, gamma-aminobutyric acid, resveratrol, polyfructose, galacto-oligosaccharides, chitosan, sodium alginate.

8. A method for the treatment of lung function anomaly caused by COVID-19 or influenza virus, including dyspnea, cough, lung nodule, lung fibrosis and lung diseases in a subject, comprising administering the Chinese medicine compositions in claim 1 to the subject in need.

9. A method for the treatment of pharyngeal diseases such as dry pharyngeal pain, pharyngeal itching and cough, sore throat, pharyngeal discomfort in a subject, comprising administering the Chinese medicine compositions in claim 1 to the subject in need.

10. A method for the treatment of the diseases caused by other influenza viruses such as avian influenza virus, hand-foot-and-mouth disease viruses in a subject, comprising administering the Chinese medicine compositions in claim 1 to the subject in need.

11. A method for the treatment of respiratory tract related diseases, such as lung grinding glass shadow, interstitial lung disease and COPD in a subject, comprising administering the Chinese medicine compositions in claim 1 to the subject in need.

12. A method for the treatment of sequelae of the lungs of patients infected with the new coronavirus or respiratory dysfunction in a subject, comprising administering the Chinese medicine compositions in claim 1 to the subject in need.

13. The method claimed in claim 6, characterized in that other nutrients can be added as auxiliary materials of the preparation to prepare different dosage forms for enhancing the effect of the above-mentioned preparation; these effective auxiliary materials are nutritional supplements consisting of at least one or two of amino acids (glycine, arginine, leucine, valine, alanine, serine acid, proline, isoleucine, phenylalanine, lysine, threonine, methionine, tryptophan, histidine, tyrosine, aspartate, cysteine, glutamic acid), curcumine or turmeric extract, betaine hydrochloride, chlorogenic acid, reduced glutathione, yeast beta-glucan, vitamin C, gamma-aminobutyric acid, resveratrol, polyfructose, galacto-oligosaccharides, chitosan, sodium alginate.
